(19) United States
(12) Patent Application Publication
(10) Pub. No.: US 2021/0100870 A1
(43) Pub. Date: Apr. 8, 2021

(54) BONE DISEASE TREATMENT

(71) Applicant: The University of Birmingham, Birmingham (GB)

(72) Inventors: George Edward Rainger, Birmingham (GB); Helen McGettrick, Birmingham (GB); Myriam Chimen, Birmingham (GB)

(21) Appl. No.: 16/948,987

(22) Filed: Oct. 8, 2020

Related U.S. Application Data
(60) Provisional application No. 62/912,439, filed on Oct. 8, 2019.
(60) Provisional application No. 63/024,218, filed on May 13, 2020.

(51) Int. Cl. A61K 38/10; A61K 35/32; A61P 19/00; A61P 19/10

(52) U.S. Cl. CPC A61K 38/10; A61P 19/10; A61P 19/00; A61K 35/32

(57) ABSTRACT

The present invention concerns methods of reducing bone loss and/or stimulating bone production comprising administering an effective amount of a peptide comprising the amino acid sequence SVTEQGAELSNEER (SEQ ID NO:1), or variants thereof, to a patient and/or bone cells. The present invention also concerns methods of treatment and/or prophylaxis of musculoskeletal loss and/or damage in a patient, comprising administering an effective amount of the peptide.

### CROSS-REFERENCE TO RELATED APPLICATIONS

This application claims the benefit of U.S. provisional application Ser. No. 62/912,439, filed 8 Oct. 2019 and U.S. provisional application Ser. No. 63/024,218, filed 13 May 2020. The entire contents of these applications are hereby incorporated by reference as if fully set forth herein.

### REFERENCE TO SEQUENCE LISTING SUBMITTED VIA EFS-WEB

This application includes an electronically submitted sequence listing in .txt format. The .txt file contains a sequence listing entitled “SequenceListing_PB157732USA” created on Oct. 7, 2020 and is 1 KB in size. The sequence listing contained in this .txt file is part of the specification and is hereby incorporated by reference herein in its entirety.

### BACKGROUND

### 1. Field of the Invention

The present invention concerns methods of reducing bone loss and/or stimulating bone production comprising administering an effective amount of a peptide comprising the amino acid sequence SVTEQGAELSNEER (SEQ ID NO:1), or variants thereof, to a patient and/or bone cells. The present invention also concerns methods of treatment and/or prophylaxis of musculoskeletal loss and/or damage in a patient, comprising administering an effective amount of the peptide.

### 2. Background of the Invention

Bones are continually replaced and remodeled throughout life in order to repair damage; maintain integrity; and to respond to changes in activity and load. Abnormalities in the bones or joints of individuals underpin pathology in musculoskeletal (MSK) diseases such as osteoporosis, cancer-induced bone disease, Paget's disease of bone and the rare groups of metabolic bone diseases; where patients suffer permanent loss of function and pain. In addition, sedentary activity where the bones are not actively loaded, such as prolonged bed-rest (>5 days) due to, for example, disease/surgery and hospitalization or space travel, leads to loss in bone mass.

Maintenance of bone integrity is a key medical challenge, especially in ageing populations. MSK diseases affect >10 million people in the UK, costing the NHS about £4.7 billion per year and accounting for over 30 million working days lost per annum (Musculoskeletal data Advisory group response to the Government's mandate to NHS England 2017/18). Existing therapies focus on reducing joint pain and/or slowing the rate of bone damage, while therapies inducing bone repair and limiting bone loss are often ignored.

Osteoporosis is the most common bone disease in the world, affecting over 44 million individuals in the US alone (Lewiecki, E. M., Clinical and Molecular Allergy 2, 2004, 9). Despite this there is no cure for osteoporosis. As such there is a clear unmet clinical need to develop novel therapies with the ability to prevent the onset of osteoporosis and more effectively treat the consequences of accelerated bone loss, namely fractures, by triggering and maintaining normal bone repair mechanisms in susceptible individuals and to cure individuals already diagnosed with osteoporosis.

Bone growth and repair is dependent predominantly on the activities of osteoblast and osteoclast cells (see Raggatt, L. J., J. Biol. Chem., 2010, 285, 25103-25108). Osteoblast cells are the major cellular component of bone and almost the entire bone matrix in a mammal is mineralized by osteoblasts. Osteoblasts synthesize and mineralize bone during both bone formation and bone remodeling. In contrast, osteoclasts break down and restructure bone tissue by producing enzymes that dissolve the collagen, calcium and phosphorus of the bone.

Currently, anti-resorptive bisphosphonates are typically used to treat osteoporosis, which inhibit bone resorption by promoting apoptosis of osteoclasts. However, long-term use is associated with increased incidence of micro-fractures and atypical femur fractures, suggesting that these drugs may hinder normal bone remodeling and repair (see, for example, Haworth, A. E. and Webb, J. Br. J. Radiol., 2002, 85(1018), 1333-1342). Newer drugs on the market include anti-RANKL antibody (denosumab); an src kinase inhibitor (saracatinib); and a cathespin K inhibitor (odanacatib), which was discontinued in 2016 due to increased risk of stroke (see Hanley, D. A. et al., Int. J. Clin. Pract., 2012, 66(12), 1139-1146; Danson, S. et al., J. Bone Oncol., 2019, 19, 100261; Bromme, D. and Lecaille, F., Expert Opin. Investig. Drugs, 2009, 18(5), 585-600). These agents help to reduce the rate of bone damage by altering the activity of osteoclasts and preventing bone resorption. However, none affect osteoblasts, the cells known to induce bone formation.

Methods of reducing bone loss and/or stimulating bone production by controlling the balance between osteoclast and osteoblast activity are likely to be useful in the treatment of MSK diseases and/or damage, including any disorder of accelerated bone loss or impaired bone remodeling, such as cancer-induced bone disease, Paget's disease of bone and the rare groups of metabolic bone diseases, or diseases associated with inflammation. Agents that stimulate bone formation, i.e. which stimulate osteoblast activity, are likely to be particularly effective in such treatment, since bone formation and mineralization would not be limited by the natural, potentially under-active activity of osteoblasts.

### SUMMARY OF THE INVENTION

Using proteomics, the inventors have identified a peptide released from B-cells after adiponectin stimulation, which they have named PEPtide Inhibitor of Trans-Endothelial Migration (PEPITEM). PEPITEM is a small peptide derived from the 14.3.3.ζδ protein by proteolytic cleavage (see Saba, J. D., Nat. Med. 2015, 21(5), 424-426 and Chimen, M. et al.,Nat. Med., 2015, 21(5), 467-480).

It has now been found that peptides comprising the PEPITEM sequence, i.e. the amino acid sequence SVTEQGAELSNEER (SEQ ID NO:1), or variants thereof, are surprisingly effective in reducing bone loss and/or stimulating bone production when administered to a patient and/or bone cells in effective amounts. Administration of such peptides to at risk patient groups has the scope to prevent bone mass loss, and in the case of an individual undergoing surgery-induced bed-rest, could significantly reduce their immobility following recovery. This is particularly important in the over 65 population, where surgery-induced bed-rest often leads to reduced independence and further incidences of illness. Further clinical applications are discussed below and may include any disorder of accelerated bone loss or impaired bone remodeling, for example cancer-induced bone disease or Paget's disease or complex fractures.

The skilled person is aware that any reference to an aspect of the invention includes every embodiment of that aspect. For example, any reference to the first aspect of the invention includes the first aspect and all embodiments of the first aspect.

Viewed from a first aspect, the invention provides a method of reducing bone loss and/or stimulating bone production, the method comprising administering an effective amount of a peptide comprising the amino acid sequence SVTEQGAELSNEER (SEQ ID NO:1, also referred to herein as PEPITEM), or variants thereof, to a patient and/or bone cells or their precursors.

Viewed from a second aspect, the invention provides a method of treatment and/or prophylaxis of musculoskeletal loss and/or damage in a patient, the method comprising administering an effective amount of the peptide of the first aspect. Viewed from a third aspect, the invention provides a composition comprising the bone cells of the first aspect, and/or their precursors, and an effective amount of the peptide of the first and second aspects. Viewed from a fourth aspect, the invention provides an orthopedic implant comprising an effective amount of the peptide of the first to third aspects of the invention. Viewed from a fifth aspect, the invention provides a composition comprising bone cement and an effective amount of the peptide of the first to fourth aspects of the invention. Viewed from a sixth aspect, the invention provides use of the peptide of the first to fifth aspects for the method of the first and second aspects. Viewed from a seventh aspect, the invention provides use of the peptide of the first to sixth aspects for the manufacture of a medicament for the method of the first and second aspects. Viewed from an eighth aspect, the invention provides an effective amount of the peptide of the first to seventh aspects for use in a method according to the first and second aspects.

Specifically, the invention relates to: a method of reducing bone loss and/or stimulating bone production, the method comprising administering an effective amount of a peptide comprising the amino acid sequence SVTEQGAELSNEER (SEQ ID NO:1), or variants thereof, to a patient in need thereof, bone cells, bone cell precursors, or a combination thereof.

Preferably, the method stimulates bone production.

In some embodiments, the method involves administration of the peptide ex vivo directly to the bone cells, bone cell precursors, surrounding media, or a combination thereof. The bone cells can be osteoblasts, for example primary osteoblasts, mammal osteoblasts, and/or human osteoblasts.

The invention also relates to methods as described above further comprising transplanting the bone cells into a patient, preferably in a patient requiring treatment and/or prophylaxis of musculoskeletal loss and/or damage.

The invention, also relates to a method of treatment, prophylaxis, or both of musculoskeletal loss or damage in a patient in need thereof, the method comprising administering an effective amount of SVTEQGAELSNEER (SEQ ID NO:1), or variants thereof. The musculoskeletal loss or damage can be associated with osteoporosis, bone injury, or both. The osteoporosis can result from any one or a combination of the group consisting of aging, prolonged bed rest, anorexia nervosa, Diabetes Mellitus (Type 1), hyperparathyroidism, inflammatory bowel disease, malabsorption, celiac disease, haemophilia, leukemias and lymphomas, multiple myeloma, lupus, rheumatoid arthritis, alcoholism, depression, emphysema, epilepsy, immobilisation, multiple sclerosis, muscular dystrophy and post-transplant bone disease. In some embodiments, the bone injury can be associated with sports injuries or any one or a combination of neurological disorders including stroke, multiple sclerosis, cerebral palsy, Parkinson's disease, spinal cord injury, neuropathy, sciatica and dementia; delirium; dizziness; vertigo; and dehydration. In some embodiments, the musculoskeletal loss and/or damage is bone fracture.

With respect to the invention, the patient preferably is a mammal, and more preferably a human.

In certain embodiments of the invention, the peptide is administered by a method selected from the group consisting of intravenous, intramuscular, intrathecal and subcutaneous administration, injection directly into a fracture, administration directly to the bone cells or surrounding media and administration by implant.

The invention also related to a composition comprising an effective amount of the peptide SVTEQGAELSNEER (SEQ ID NO:1) or a variant thereof and bone cells or bone cell precursors, and to an orthopedic implant comprising an effective amount of this composition. The invention also comprises a composition comprising bone cement and an effective amount of the peptide SVTEQGAELSNEER (SEQ ID NO:1) or a variant thereof.

### BRIEF SUMMARY OF THE DRAWINGS

FIG. 1A is a set of images of MC3T3 cells at the indicate days 16, 18, and 20, in the indicated media.

FIG. 1B, FIG. 1C, and FIG. 1D are images of primary murine osteoblast cells at day 12 in the indicated media.

FIG. 1E and FIG. 1F are bar graphs showing alizarin red concentration of treated and untreated murine osteoblast cell line MC3T3 and primary murine osteoblasts, respectively.

FIG. 1G, FIG. 1H, and FIG. 1I are bar graphs showing alkaline phosphatase activity in human osteoblasts.

FIG. 2A and FIG. 2B show microCT images of phosphate buffered saline (PBS)- and PEPITEM-treated long bones of mice, respectively.

FIG. 2C, FIG. 2D, FIG. 2E, and FIG. 2F show quantitated data on bone volume/trabecular volume, trabecular number, trabecular thickness, and trabecular separation, respectively.

FIG. 3A and FIG. 3B show microCT images of mouse vertebra after treatment with PBS (control) and PEPITEM, respectively.

FIG. 3C, FIG. 3D, FIG. 3E, and FIG. 3F show quantitated data on bone volume/trabecular volume, trabecular number, trabecular thickness, and trabecular separation, respectively.

FIG. 4A is a photograph of the apparatus for 3-point bending tests of mouse long bones.

FIG. 4B is a graph of force versus displacement, showing stiffness and failure.

FIG. 4C, FIG. 4D, and FIG. 4E are graphs showing data for stiffness, bending force, and fracture force, respectively.

FIG. 5A is a set of microCT images of mouse long bones at baseline, and after treatment with PBS (negative control) or PEPITEM for four weeks.

FIG. 5B, FIG. 5C, and FIG. 5D show data for bone volume/trabecular volume, trabecular number, and trabecular separation, respectively.

FIG. 6A and FIG. 6B are tartrate-resistant acidic phosphate (TRAP) stained sections of decalcified tibia (treated with PBS and PEPITEM, as indicated) with the region of interest (ROI), used to calculate osteoclast numbers, and the line measurements, used to calculate chondroclast numbers, shown in red.

FIG. 6C and FIG. 6D are histograms showing data on osteoclast and chondroclast numbers in these mice.

FIG. 7 presents data for murine osteoclast precursor cells, were cultured in wells within an osteoassay plate, in the absence (−) or presence of osteoclastogenic media (+; differentiation) with (+) or without (−) 10 ng/ml of PEPITEM.

### DETAILED DESCRIPTION

### 1. Overview

The peptides of the invention, and the associated methods and uses, are surprisingly effective in reducing bone loss and/or stimulating bone production when administered to a patient and/or bone cells in effective amounts. Consequently, the peptides of the invention are useful in methods of reducing bone loss and/or stimulating bone production. Such methods may be used to treat patients suffering from bone damage, weakening and/or degeneration. Therefore, the peptides of the invention are useful in the treatment and/or prophylaxis of musculoskeletal loss and/or damage. The peptide, methods and uses of the invention are described in detail below.

### 2. Summary of Results

Further discussion of the figures and results therein is contained below.

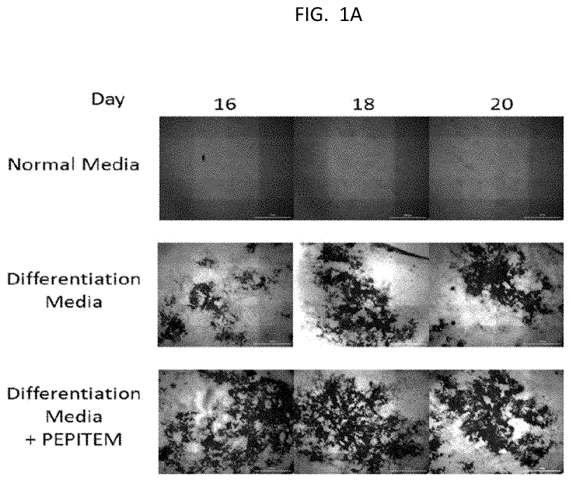

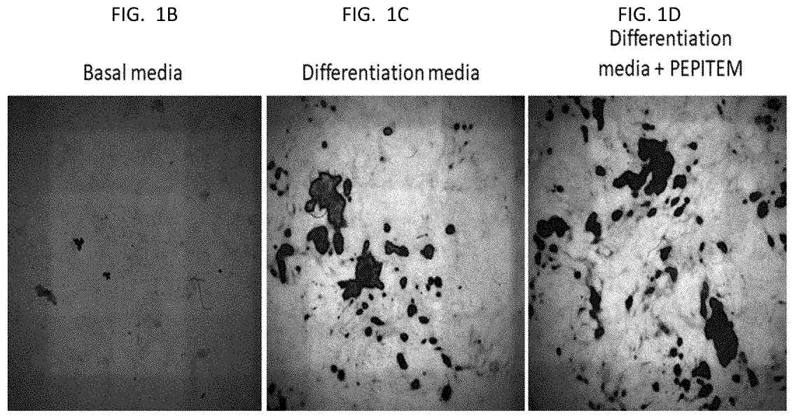

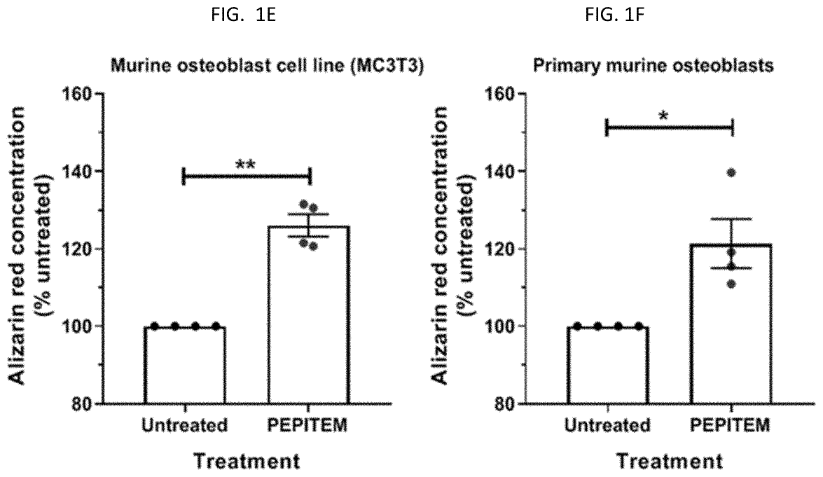

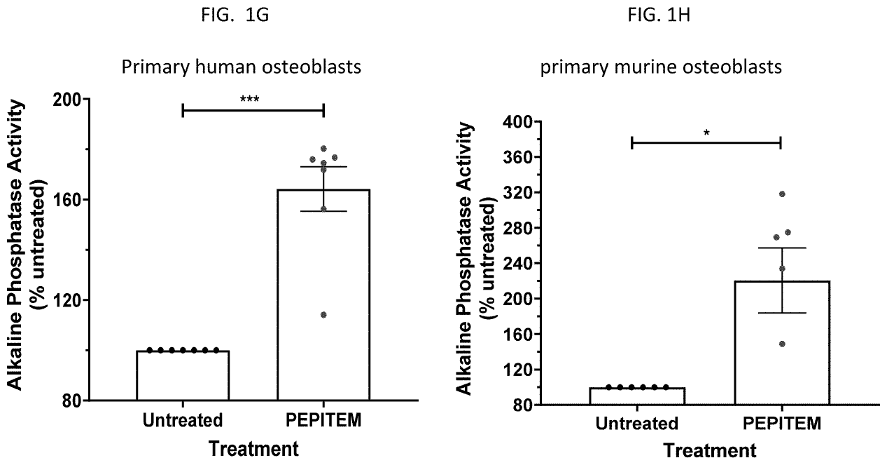

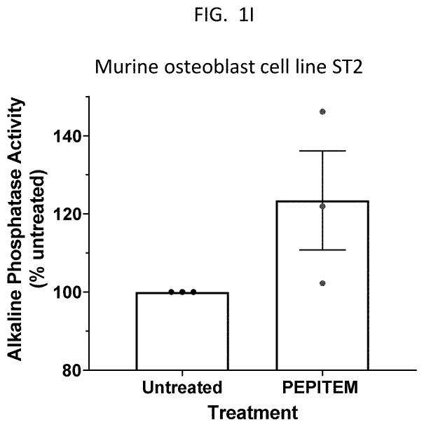

For the data presented in FIG. 1, the murine osteoblast cell line MC3T3 (FIG. 1E), primary murine osteoblast cells (FIG. 1F) or primary human osteoblasts (FIG. 1G) were allowed to mineralize over 21 days in the presence or absence of PEPITEM. Mineralization, as a measure of bone formation, was assessed by quantification of Alizarin Red staining in murine osteoblasts using colorimetric spectrometry. The images in FIG. 1A show MC3T3 cells at days 16, 18 and 20. The images in FIG. 1B, FIG. 1C, and FIG. 1D show primary murine osteoblast cells at day 12. The alkaline phosphatase activity in murine osteoblast cell line, primary murine osteoblasts, and human osteoblasts (see FIG. 1E, 1F, and 1G) shows that PEPITEM significantly increased murine and human primary osteoblast mineralization. *=p<0.05 and **=p<0.01 by paired t-test compared to untreated cells.

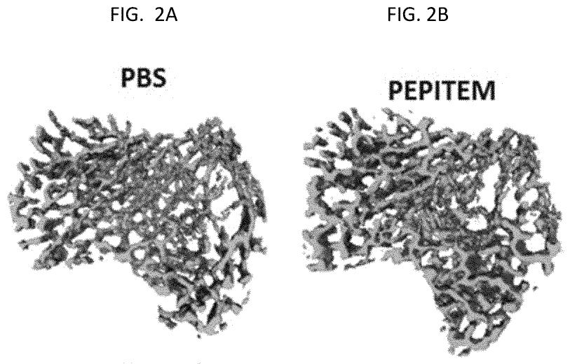

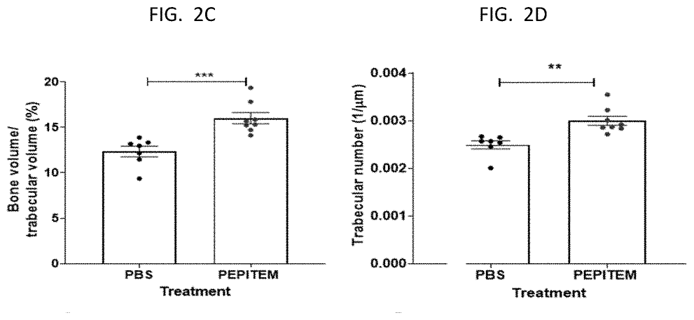

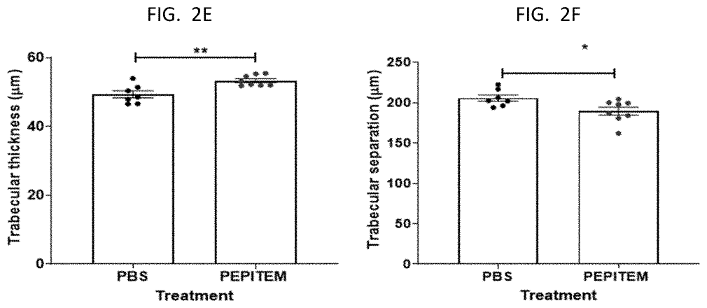

For the data presented in FIG. 2, young, healthy wild-type mice were given daily injections with either PBS or PEPITEM for 14 days. MicroCT images were obtained from the long bones (see FIG. 2A and 2B). The microCT data quantitated in FIG. 2C-FIG. 2F show that PEPITEM significantly increases trabecular bone formation compared to PBS on treatment over two weeks; *=p<0.05, **=p<0.01 and ***=p<0.001 by unpaired t-test. PEPITEM significantly increased the bone volume to trabecular bone volume ratio (BV/TV) (FIG. 2C), trabecular number (FIG. 2D), and trabecular thickness (FIG. 2E), and decreased trabecular separation (FIG. 2F).

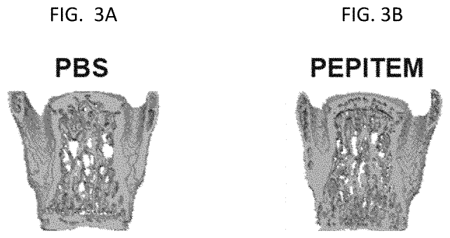

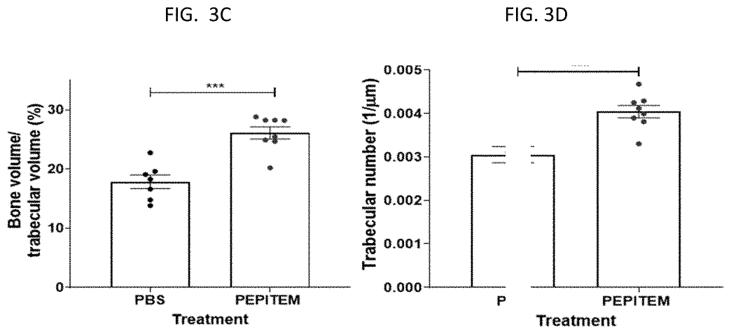

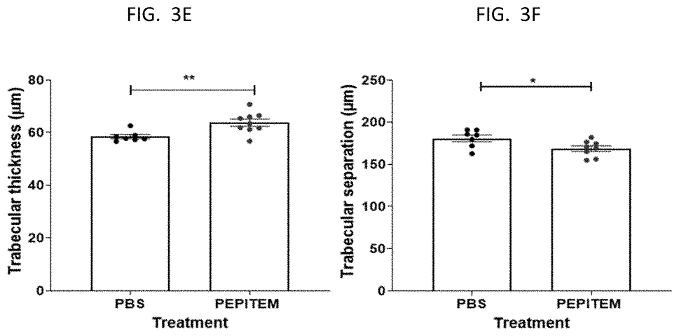

For the data presented in FIG. 3, young, healthy wild-type mice were given daily injections with either PBS or PEPITEM for 14 days. MicroCT images were obtained from the vertebra (see FIG. 3A and FIG. 3B). The quantitated data in FIG. 3C through FIG. 3F show that PEPITEM significantly increases trabecular bone formation compared to PBS on treatment over two weeks; *=p<0.05, **=p<0.01 and ***=p<0.001 by unpaired t-test. PEPITEM significantly increased the bone volume to trabecular bone volume ratio (BV/TV) (FIG. 3C); trabecular number (FIG. 3D), and trabecular thickness (FIG. 3E), and decreased trabecular separation (FIG. 3F).

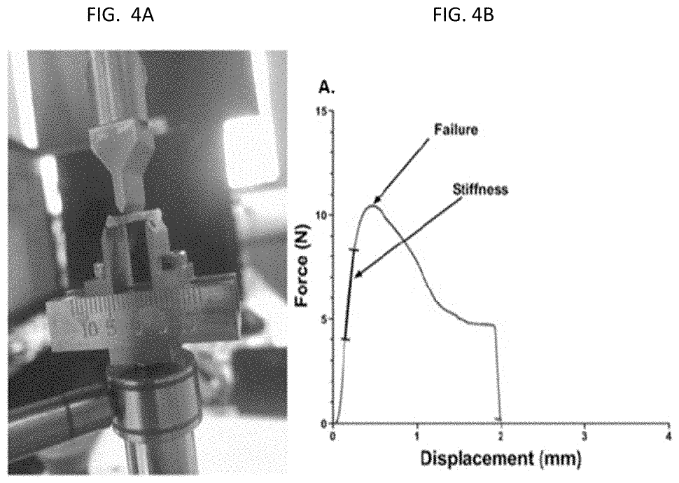

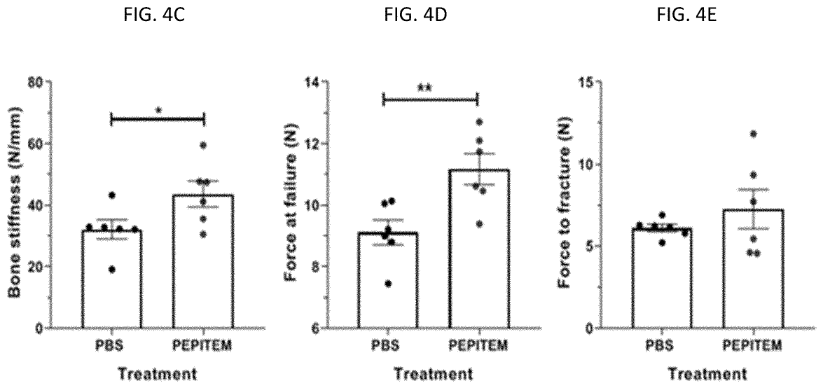

For the data presented in FIG. 4, young, healthy wild-type mice were given daily injections with either PBS or PEPITEM for 14 days. Long bones were subjected to a 3-point bending test ex vivo (see FIG. 4A and FIG. 4B) to measure the stiffness of the bone, the force required to induce the bone to bend and the force required to completely fracture/break the bone. These data are presented in FIG. 4C through FIG. 4E. PEPITEM significantly increased the stiffness (FIG. 4C), bending force (FIG. 4D) and the fracture force (FIG. 4E) of the long bones. PEPITEM significantly increases the strength of long bones over a two-week treatment period.

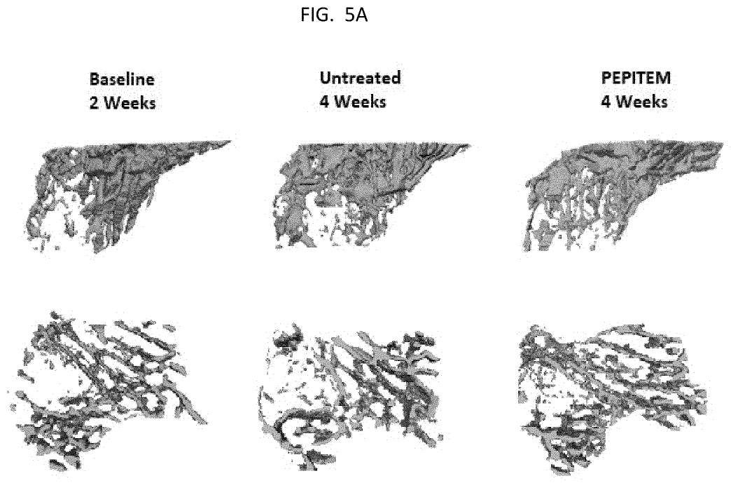

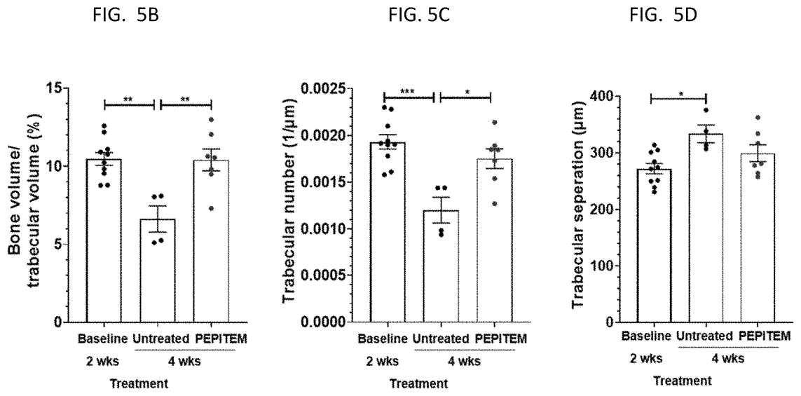

For the data presented in FIG. 5, young, healthy wild-type mice were subjected to an ovariectomy. After 2 weeks, the mice were culled for baseline bone analysis, and either left untreated for 2 weeks or given daily injections with PEPITEM for 2 weeks. MicroCT images were obtained from the long bones either 2- or 4-weeks post ovariectomy (see FIG. 5A). The quantitation of the data show that PEPITEM significantly increased the bone volume to trabecular bone volume ratio (BV/TV) (FIG. 5B); trabecular number (FIG. 5C) and decreased the trabecular separation (FIG. 5D). PEPITEM prevented additional bone loss when compared to 2-week untreated mice.

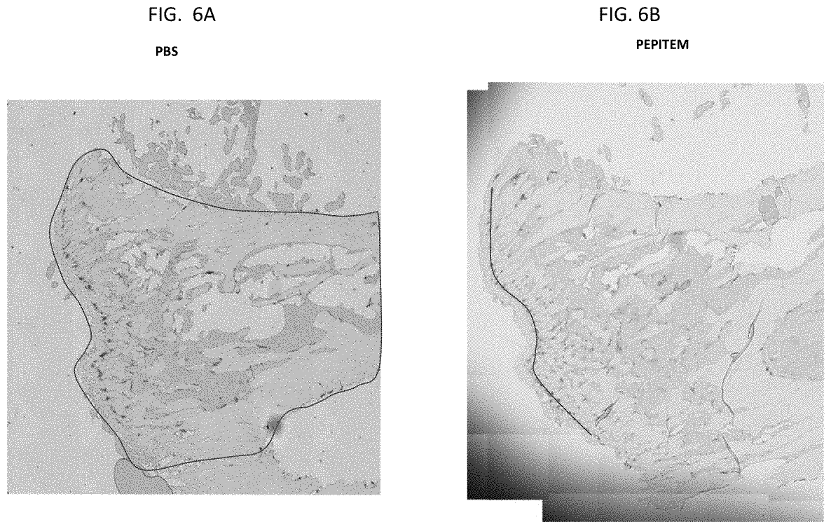

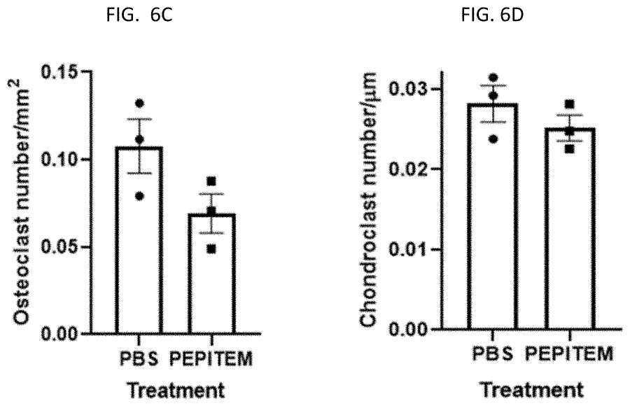

For the data presented in FIG. 6, young, healthy wild-type mice were given daily injections with either PBS or PEPITEM for 14 days, after which sections of decalcified tibia were analyzed by tartrate-resistant acidic phosphatase (TRAP) staining to calculate the number of osteoclasts and chondroclasts in each section. FIG. 6A through FIG. 6B show images of tibia diaphysis sections with the region of interest (ROI), used to calculate osteoclast numbers, and the line measurements, used to calculate chondroclast numbers, shown in red. FIG. 6C and FIG. 6D are histograms showing a decrease in osteoclast and chondroclast numbers on treatment of mice with PEPITEM.

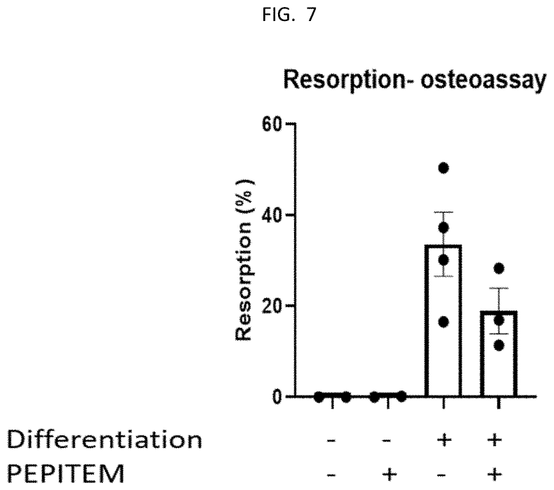

For the data presented in FIG. 7, murine osteoclast precursor cells were cultured in wells, within an osteoassay plate, in the absence (−) or presence of osteoclastogenic media (+; differentiation) with (+) or without (−) 10 ng/ml of PEPITEM. Cells differentiated at around day 7. The surface of the wells were analyzed for osteoclast resorption activity by removing the cells and imaging any pits or multiple pit clusters on the well surface. The histogram shows that osteoclast resorption increased when cells were cultured with PEPITEM.

### 3. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the discussion that follows, reference is made to a number of terms, which have the meanings provided below, unless a context indicates to the contrary. The nomenclature used herein for defining compounds, in particular the compounds according to the invention, is in general based on the rules of the IUPAC organization for chemical compounds, specifically the “IUPAC Compendium of Chemical Terminology (Gold Book)”.

The term “about,” as used herein, means plus or minus 20 percent of the recited value, so that, for example, “about 0.125” means 0.125±0.025, and “about 1.0” means 1.0±0.2.

As used herein, the term “comprising” or variants thereof will be understood to imply the inclusion of a stated element, integer or step, or group of elements, integers or steps, but not the exclusion of any other element, integer or step, or group of elements, integers or steps.

As used herein, the term “consisting” or variants thereof will be understood to imply the inclusion of a stated element, integer or step, or group of elements, integers or steps, and the exclusion of any other element, integer or step or group of elements, integers or steps.

As used herein, the term “consists essentially of,” when used in reference to sequences of amino acids or nucleotides, means a sequence that the sequence contains no more than one or two deletions, additions, or substitutions from the base sequence. Preferably, any substitutions are conservative substitutions.

As used herein, the term “stereoisomer” is used herein to refer to isomers that possess identical molecular formulae and sequence of bonded atoms, but which differ in the arrangement of their atoms in space.

As used herein, the term “diastereoisomers” (also known as diastereomers) defines stereoisomers that are not related as mirror images.

As used herein, the term “enantiomer” defines one of a pair of molecular entities that are mirror images of each other and non-superposable, i.e. cannot be brought into coincidence by translation and rigid rotation transformations. Enantiomers are chiral molecules, i.e. are distinguishable from their mirror image.

As used herein, the term “racemic” is used herein to pertain to a racemate. A racemate defines a substantially equimolar mixture of a pair of enantiomers.

As used herein, the term “isotope” is used herein to define a variant of a particular chemical element, in which the nucleus necessarily has the same atomic number but has a different mass number owing to it possessing a different number of neutrons.

As used herein, the term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

As used herein, the term “biocompatible” is used herein to refer to a material that is not harmful or toxic to living tissue.

As used herein, the term “treatment” defines the therapeutic treatment of a human or non-human animal patient, in order to impede or reduce or halt the rate of the progress of the condition, or to ameliorate or cure the condition. Prophylaxis of the condition as a result of treatment is also included. References to prophylaxis are intended herein not to require complete prevention of a condition: its development may instead be hindered through treatment in accordance with the invention. Typically, treatment is not prophylactic, and the compound or composition is administered to a patient having a diagnosed or suspected condition.

As used herein, the term “effective amount” herein defines an amount of the compound or composition of the invention that is sufficient to impede the noted diseases and thus produces the desired therapeutic or inhibitory effect.

As used herein, the term “prodrug” is used herein to refer to a compound which acts as a drug precursor and which, upon administration to a subject, undergoes conversion by metabolic or other chemical processes to yield a compound of formula (I).

As used herein, the term “pharmaceutically acceptable excipient” defines substances other than a pharmacologically active drug or prodrug, which are included in a pharmaceutical product.

As used herein, the term “intrathecal administration” defines administration of a compound by injection into the spinal canal, or into the subarachnoid space.

As used herein, the term “intraosseous administration” defines administration of a compound by injection into the bone marrow.

As used herein, the term “intravenous administration” defines administration of a compound by injection into a vein or veins.

As used herein, the term “intramuscular administration” defines administration of a compound by injection into a muscle.

As used herein, the term “subcutaneous administration” defines administration of a compound by injection into the subcutis, i.e. the layer of skin directly below the dermis and epidermis.

As used herein, the term “oral administration” defines administration of a compound through the mouth, wherein the compound is typically in the form of a tablet or capsule.

As used herein, the term “variant thereof,” in the context of a sequence of amino acids or nucleotides refers to sequences that are highly similar to the base sequence. For example, a sequence with a 80%, 85%, 90%, 95%, 96%, 97% identical sequence, including 12 out of 14 identical amino acids. Variants of a sequence can include any sequence with deletions, additions, or substitutions (replacements) to the original sequence. Preferably, any substitutions are conservative substitutions. Where the amino acid sequence is situated at either end of the peptide, variants also comprise the amino acid sequence modified at the N- or C-terminus with a chemical moiety.

As used herein, the term “prolonged bed rest” is used herein to refer to bed rest for a period of time ranging from several days to several months. The skilled person is aware that a patient is not necessarily immobile for the entirety of the period or confined to bed because of a health impairment that physically prevents them from leaving bed. However, the patient is necessarily in bed for the majority of the period.

### 4. Embodiments of the Invention

The method of the first aspect of the invention is a method of reducing bone loss and/or stimulating bone production. Bone loss and production are dependent on the balance of osteoclast and osteoblast activities. If the activities are such that the rate of bone cell generation is greater than the rate of bone cell resorption then there is an overall production of bone. If the rate of bone cell resorption is greater than the rate of bone cell generation then there is overall bone loss. On the other hand, if the rates of bone cell production and bone cell resorption are approximately equal then the amount of bone is approximately constant.

Bone is herein defined to be any type of bone tissue, i.e. cortical bone tissue, cancellous bone tissue and/or bone marrow. All of these tissues are formed by osteoblasts, which produce the protein osteoid, which mineralizes to become bone. Bone cells are defined to be any type of cell found in bone, including osteoblasts, osteoclasts and osteocytes. Osteocytes are derived from osteoblasts and contribute to bone regeneration by directing osteoclasts to sites in need of repair.

MSK diseases such as osteoporosis, in which bones weaken and become more brittle, reflect a relative enhancement of osteoclast activity such that the rate of bone cell resorption is greater than bone cell generation. Thus, osteoclasts are a prominent therapeutic target, and their inhibition or apoptosis is the mechanism of action of the commonly-used bisphosphonate MSK agents. However, long-term use of bisphosphonates is associated with increased incidence of micro-fractures and atypical femur fractures, suggesting that these drugs may hinder normal bone remodeling and repair. Furthermore, use of bisphosphonates in children has in some cases induced osteopetrosis, in which bones become abnormally dense and prone to fracture, see S. L. Teitelbaum, Am. J. Pathol., 2007, 170(2), 427-435.

In contrast, in addition to inhibiting osteoclast production, the peptides of the invention stimulate the production of bone by osteoblasts, thereby increasing the rate of bone formation relative to bone resorption with the result that bone loss is reduced and, when the rate of bone formation is increased such that it is greater than the rate of bone resorption, bone is produced. Therefore, the methods of the invention are not limited by the inherent and potentially under-active activity of the osteoblast cells that are treated.

The methods of the invention comprise administering an effective amount of the peptide. “Effective amount” is used herein to refer to concentrations of the peptide that lead to an enhanced rate of bone formation relative to bone resorption. The skilled person is aware that the effective amount of peptide is not restricted to amounts that lead to overall bone production. Rather, the effective amount includes amounts that reduce the rate of bone loss. The skilled person is further aware that an effective amount is likely to vary with the particular compound of the invention, the subject and the administration procedure used. It is within the means and capacity of the skilled person to identify the effective amount of the compounds and compositions of the invention via routine work and experimentation. Typically, the effective amount lies within a range of 1 mg/kg to 100 mg/kg.

The peptide of the invention comprises the amino acid sequence SVTEQGAELSNEER (SEQ ID NO:1), or variants thereof. Variants of the amino acid sequence are envisaged, provided that such variants are able to reduce bone loss and/or stimulate bone production. Variants may have an improved ability to reduce bone loss and/or stimulate bone production. This may be through changes in affinity for cognate receptor(s) or changes that alter the pharmacokinetic profile of the peptide in vivo. It will be appreciated that it is now within the skill of the art to modify peptide chemistry to increase the pharmacological ‘profile’ of peptides in vivo, and that these changes are not based solely on amino acid substitution. Variants include peptides comprising a version of the amino acid sequence SVTEQGAELSNEER (SEQ ID NO:1) in which one or more amino acids, for example 1, 2, 3 or 4 amino acids, have been altered, either by deletion or substitution. Alternatively, the amino acid sequence may be altered by the addition of one or more amino acids, for example 1 to 6 amino acids, for example 1, 2, 3, 4, 5 or 6 amino acids.

In some embodiments, the variants comprise the amino acid sequence altered by substitution of one or more amino acids for another. The substitution may be a conservative replacement, by which is meant that any given amino acid is replaced by a different amino acid with similar biochemical properties. For example, where the amino acid is a serine or threonine, it may be replaced with a different amino acid selected from the group consisting of serine, cysteine, selenocysteine, threonine and methionine. Where the amino acid is a valine, glycine, alanine or leucine, it may be replaced with a different amino acid selected from the group consisting of glycine, alanine, valine, leucine and isoleucine. Where the amino acid is arginine, it may be replaced with a different amino acid selected from the group consisting of histidine and lysine. Finally, where the amino acid is a glutamate, asparagine or a glutamine, it may be replaced with a different amino acid selected from the group consisting of aspartate, glutamate, asparagine and glutamine.

The preferred peptide is 14 amino acids long, although the peptide can also be as few as 13, 12, 11 or 10 amino acids or as many as 15, 16, 17 18, 19 or 20 amino acids. Where amino acids are added or removed, these are preferably to or from the N and/or C terminus of the peptide.

Where the amino acid sequence is situated at either end of the peptide, variants also comprise the amino acid sequence modified at the N- or C-terminus with a chemical moiety. In some embodiments, the N-terminus of the peptide is modified such that one of the proton atoms bound to the nitrogen atom of the amino moiety is replaced with any one of the group consisting of acetyl, propionyl, myristoyl, palmitoyl, ubiquityl, biotinyl, dansyl, 2,4-dinitrophenyl, fluorescein, 7-methoxycoumarin acetic acid, and palmitic acid. In some embodiments, the C-terminus of the peptide is modified such that the hydroxy group bound to the carbon atom of the carboxylic acid moiety is replaced with an amino group, thereby forming an amide. Other modifications to the chemical structure that protect the peptide from degradation or clearance in vivo are also preferred variants, for example, PEGylation which utilizes a linker or spacer as is known in the art.

In some embodiments, the peptide comprises the amino acid sequence SVTEQGAELSNEER (SEQ ID NO:1). In other embodiments, the peptide is PEPITEM or variants thereof, i.e. it consists of the amino acid sequence SVTEQGAELSNEER (SEQ ID NO:1), or variants thereof. In some embodiments, the peptide consists essentially of the amino acid sequence SVTEQGAELSNEER (SEQ ID NO:1). In further embodiments, the peptide is PEPITEM, i.e. the peptide consists of the amino acid sequence SVTEQGAELSNEER (SEQ ID NO:1).

“Consists essentially of,” or “variants thereof,” therefore when used in the context of PEPITEM, refers to amino acid sequences sharing the same sequence as 12 or more of the amino acids of SVTEQGAELSNEER (SEQ ID NO:1). For example, any one or two of the amino acids of this sequence may be deleted or replaced with any one or two different amino acids. Where the amino acids are replaced with different amino acids, it will typically be a conservative replacement.

The methods of the invention comprise administering an effective amount of the peptide of the invention to a patient and/or bone cells and/or their precursors. The patient may be any animal comprising a skeleton made of bone. In some embodiments, the patient is any one of the group consisting of mammal, bird, reptile and amphibian. In other embodiments, the patient is a mammal. In some embodiments, the patient is any one selected from the group consisting of human, horse, dog, cattle, goat, sheep, pig, cat, bison, camel, llama and alpaca. In more specific embodiments, the patient is any one selected from the group consisting of human, horse, dog, cattle, goat, sheep, pig and cat, most often a human.

The bone cells comprise any one or a selection from the group consisting of osteoblasts, osteoclasts and osteocytes or their precursors. In some embodiments the bone cells consist primarily of osteoblasts and/or osteoblast precursors. In further embodiments, the bone cells are osteoblasts. The inventors have identified that the receptor for the peptide of the invention is surprisingly expressed by osteoblasts.

In some embodiments, the peptide of the invention is administered to a patient and/or bone cells. In these embodiments, the peptide of the invention is not administered to the precursors of the bone cells.

In one embodiment, the method of the invention is a method of stimulating bone production, the method comprising administering an effective amount of the peptide of the invention to a patient and/or bone cells and/or their precursors. In this embodiment, the rate of bone cell generation is greater than the rate of bone cell resorption such that there is an overall production of bone. Methods of stimulating bone production may find use in the treatment or prophylaxis of any of the conditions described herein. In addition, stimulating bone production may find use in dentistry and orthodontistry, in which any treatment requiring jaw bone growth and/or repair may benefit from application of an effective amount of the peptide of the invention. Such treatments include tooth and jaw alignment.

In specific embodiments, the peptide of the invention is administered ex vivo, i.e. in or on tissue in an external environment, outside of the patient. In such embodiments, the peptide is administered directly to bone cells and/or their precursors or surrounding media. Typically, the bone cells are osteoblasts. Often, the osteoblasts are primary osteoblasts, i.e. osteoblasts that are taken directly from living tissue and established for growth in vitro. In some embodiments the osteoblasts are derived from any one of the group consisting of a mammal, bird, reptile and amphibian. Typically, the osteoblasts are derived from a mammal. Preferably the osteoblasts are derived from a human.

In some embodiments, the peptide is administered ex vivo directly to the bone cells and/or their precursors or surrounding media. In some embodiments the peptide is administered directly to the bone cells or surrounding media, and not precursors of the bone cells. The bone cells may be primary bone cells derived from the patient. Alternatively, they may be derived from (their precursors may be) any one of the group consisting of mesenchymal stem cells, pluripotent stem cells, induced pluripotent stem cells, and peripheral blood mononuclear cells. Where the bone cells are derived from stem cells, they are typically derived from mesenchymal stem cells taken from bone marrow. For further information on the common types and sources of stem cells available, see Zakrzewski, W., Stem Cell. Res. Ther., 2019, 10(68), 1-22. Bone cells may be prepared from stem cells in vitro by manipulating culture conditions, thereby restricting differentiation to specific pathways. For a review of in vitro directed differentiation see Cohen, D. E., Melton, D., Nat. Rev. Genet., 2011, 12, 243-252. The skilled person is aware of the conditions required to promote differentiation of stem cells to bone cells. Typically, osteoblasts are cultured in mineralization differentiation media and osteoclasts are cultured in osteoclastogenic media.

Thus in a further aspect, the invention provides a composition comprising the bone cells and/or their precursors and the peptide of the invention. In some embodiments, the composition comprises the bone cells of the invention, i.e. not the precursors of the bone cells, and the peptide of the invention.

In certain embodiments, where the peptide is administered ex vivo onto the bone cells, the bone cells are then transplanted into a patient. The bone cells may be cultured for a specific time in vivo before transplant, or the bone cells may be transplanted immediately following ex vivo administration of the peptide. In such embodiments, the bone cells are transplanted conjunctly with the peptide. In other embodiments, the bone cells are transplanted consecutively or separately to the peptide.

The patient may be as described above. The bone cells may be transplanted into the patient by any one or a combination of the methods consisting of intrathecal and intraosseous injection, injecting the cells directly into a fracture and administering the cells directly to the bone or surrounding media (for example, in open surgery). Bone formation by transplanted human osteoblasts has been reported by Yamanouchi, K. et al., J. Bone Miner. Res., 2001, 16(5), 857-867 and accelerated bone fracture healing as a result of transplanted osteoblasts has been reported by Kim, S-J et al., BMC Musculoskelet. Disord., 2009, 10(20), 1-9.

In some embodiments, where the peptide is administered ex vivo onto the bone cells, and the bone cells are then transplanted into a patient, the patient requires treatment and/or prophylaxis of musculoskeletal loss and/or damage.

Viewed from a further aspect, the invention provides a method of treatment and/or prophylaxis of musculoskeletal loss and/or damage in a patient, the method comprising administering an effective amount of the peptide of the invention.

In some embodiments of the invention, musculoskeletal loss and/or damage is associated with osteoporosis and/or bone injury. For a detailed review of the primary and secondary causes of osteoporosis, see Chapter 3: Diseases of Bone of the U.S. Department of Health and Human Services. Bone Health and Osteoporosis: A Report of the Surgeon General. Rockville, Md.: U.S. Department of Health and Human Services, Office of the Surgeon General, 2004, included herein by reference. In some embodiments, said osteoporosis results from any one or a combination of the group consisting of aging; prolonged bed rest; space travel; genetic disorders including cystic fibrosis, Ehlers-Danlos, glycogen storage diseases, Gaucher's disease, homocystinuria, hypophosphatasia, idiopathic hypercalciuria, Marfan syndrome, Menkes steely hair syndrome, osteogenesis imperfect, porphyria and Riley-Day syndrome; hypogonadal states including androgen insensitivity, anorexia nervosa, athletic amenorrhea, hyperprolactinemia, panhypopituitarism, premature ovarian failure and Turner's and Klinefelter's syndrome; endocrine disorders including acromegaly, adrenal insufficiency, Cushing's syndrome, diabetes mellitus (Type 1), hyperparathyroidism and thyrotoxicosis; gastrointestinal diseases including gastrectomy, inflammatory bowel disease, malabsorption, celiac disease and primary biliary cirrhosis; hematologic disorders including haemophilia, leukemias and lymphomas, multiple myeloma, sickle cell disease, systemic mastocytosis and thalassemia; alcoholism; amyloidosis; chronic metabolic acidosis; congestive heart failure; depression; emphysema; end stage renal disease; epilepsy; idiopathic scoliosis; immobilization; multiple sclerosis; muscular dystrophy; post-transplant bone disease; and sarcoidosis.

In some embodiments, said osteoporosis results from any one or a combination of the group consisting of aging, prolonged bed rest, anorexia nervosa, Diabetes Mellitus (Type 1), hyperparathyroidism, inflammatory bowel disease, malabsorption, celiac disease, hemophilia, leukemias and lymphomas, multiple myeloma, lupus, alcoholism, depression, emphysema, epilepsy, immobilization, multiple sclerosis, muscular dystrophy and post-transplant bone disease.

In more specific embodiments, said osteoporosis results from any one or a combination of the group consisting of aging, prolonged bed rest, anorexia nervosa, hyperparathyroidism, hemophilia, leukemias and lymphomas, multiple myeloma, alcoholism, depression, emphysema, epilepsy, immobilization, muscular dystrophy and post-transplant bone disease.

In specific embodiments, the osteoporosis results from aging, i.e. is age-related osteoporosis. In other more specific embodiments, musculoskeletal loss and/or damage is associated with age-related osteoporosis.

In some embodiments, when musculoskeletal loss and/or damage is associated with osteoporosis, it is typically bone fracture or break. In other embodiments, musculoskeletal loss and/or damage is fracture, typically of the hip.

In some embodiments, the bone injury is associated with sports injuries, or is associated with any one or a combination of neurological disorders including stroke, multiple sclerosis, cerebral palsy, Parkinson's disease, spinal cord injury, neuropathy, sciatica and dementia; delirium; dizziness; vertigo; and dehydration.

In specific embodiments, the bone injury is break or fracture.

The peptide of the invention may be administered by any one of the methods consisting of intravenous, intramuscular, intrathecal, intraosseous, subcutaneous and oral administration, injection directly into a fracture, administration directly to the bone cells or surrounding media and administration by implant. In some embodiments, the peptide is administered by any one of the methods consisting of intravenous, intramuscular, intrathecal and subcutaneous administration, injection directly into a fracture, administration directly to the bone cells or surrounding media and administration by implant.

In one embodiments, the peptide is administered orally, for example in tablet form, or by injection.

“Implant” is used herein to refer to any biocompatible device for insertion into the patient, and which releases the peptide into its surrounding area. Such devices are particularly useful for controlled and/or sustained peptide release. Effective amounts of peptide may be released from such a device for a period of several hours to several years. For a review of drug-releasing implants, see Santos, A. et al., J. Mater. Chem. B, 2014, 2, 6157-6182 and Stewart, S. A. et al., Polymers (Base1), 2018, 10(12), 1379. The skilled person is aware that the rate of peptide release from an implant is dependent on the materials used to form the implant and the flow of bodily fluids surrounding the implant. For example, the implant may comprise membranes that are semi-permeable to the peptide and thus delay or decrease the rate of peptide release.

Thus, viewed from a further aspect, the invention provides an orthopedic implant comprising the peptide of the invention and in some embodiments peptide release from the orthopedic implant is sustained for a period of several hours to several years.

The orthopedic implant of the invention may or may not be biodegradable. Where the implant is biodegradable, it may be formed from one or a combination of the materials consisting of poly(lactic acid), poly(glycolic acid), poly(lactic-co-glycolic acid), poly(caprolactone), poly(amides), poly(anhydrides), poly(phosphazenes), poly(dioxanone), silk, cellulose and chitosan. Where the implant is non-biodegradable, it may be formed from one or a combination of the materials consisting of poly(siloxanes), poly(ethylene-vinyl acetate) and poly(urethanes).

The orthopedic implant may comprise a polymer coating, and in some embodiments the implant comprises the peptide of the invention within a coating. The coating may be contacted with surgical hardware, including surgical plates, rods, pins, wires, washers, nails and screws that are typically used to repair bone damage (see Nguyen, V. D. and London, J. Radiology, 1986, 158, 129-131). Thus, in some embodiments, the orthopedic implant of the invention comprises orthopedic hardware coated with the peptide. Such an implant may achieve a sustained release of the peptide in a localized area of bone damage. For a review of bioactive coatings of orthopedic implants, see Zhang, B. G. X et al., Int. J. Mol. Sci., 2014, 15(7), 11878-11921 and Goodman, S. B., Keeney, Y. Z. and Yang F., Biomaterials, 2013, 34(13), 3174-3183.

Commonly, orthopedic implants are fixed into place using bone cement. Thus, viewed from a further aspect, the invention provides a composition comprising bone cement and the peptide of the invention. In some embodiments, the bone cement is selected from any one of the group consisting of polymethyl methacrylate, calcium phosphate cement and glass polyalkenoate cement. For a review on bone cement, see Vaishya, R., Chauhan, M. and Vaish, A., J. Clin. Orthop. Trauma, 2013, 4(4), 157-163.

When the peptide of the invention is administered by implant, it is typically administered as a single dose. However, replacement of the implant and further dose administration are included within the scope of the invention. When the peptide is administered by other means, it may be administered in one or more doses per one or more day(s). For example, the peptide may be administered in a single dose every day, week, fortnight or month, with the dose reducing or stopping on recovery of the bone.

Viewed from further aspects, the invention provides:
use of a peptide of the invention for a method of the invention;
use of a peptide of the invention for the manufacture of a medicament for a method of the invention; and
a peptide of the invention for use in a method of the invention.

The peptide of the invention may be in the form of a pharmaceutically acceptable salt. The term “pharmaceutically acceptable salt” is intended to define organic and/or inorganic salts that are pharmaceutically useful. The peptide may be isolated from reaction mixtures as a pharmaceutically acceptable salt. Alternatively, the pharmaceutically acceptable salt may be prepared in situ during the final isolation and purification of the peptide by reaction with a suitable base such as a hydroxide, carbonate or bicarbonate of a pharmaceutically acceptable metal cation, or with ammonia or a primary, secondary or tertiary amine. Pharmaceutically acceptable salts include cations based on alkali metals or alkaline earth metals such as lithium, sodium, potassium, calcium, magnesium and aluminum salts and nontoxic quaternary ammonia and amine cations including ammonium, tetramethylammonium, tetraethylammonium, methylamine, dimethylamine, trimethylamine, triethylamine, diethylamine, and ethylamine. Other examples of organic amines useful for the formation of base addition salts include ethylenediamine, ethanolamine, diethanolamine, piperidine, and piperazine.

The pharmaceutically acceptable salt may also be prepared by treatment of the peptide with a suitable acid, for example, hydrogen chloride, hydrogen bromide, hydrogen iodide, sulfuric acid, phosphoric acid, acetic acid, trifluoroacetic acid, propionic acid, glycolic acid, maleic acid, malonic acid, methanesulfonic acid, fumaric acid, succinic acid, tartaric acid, ciric acid, benzoic acid and ascorbic acid.

The skilled person is aware that all naturally occurring amino acids with chiral carbon centres are formed in the L-configuration (levorotatory), with the exception of glycine, which has no chiral carbon center. Therefore, when prepared from naturally-occurring amino acids, the peptide of the invention exists in one enantiomeric form, in which all amino acids are in the L-configuration. However, unnatural amino acids with chiral carbon centers may exist in the D-configuration (dextrorotatory) or in mixtures of both the L- or D-configuration. Therefore, when prepared from any unnatural amino acids, the peptide of the invention may exist in different enantiomeric forms. All enantiomers, diastereoisomers and racemic mixtures, are included within the scope of the invention. Individual stereoisomers of the peptide of the invention, i.e., associated with less than 5%, preferably less than 2% and in particular less than 1% of the other stereoisomer, are included. Mixtures of stereoisomers in any proportion, for example a racemic mixture comprising substantially equal amounts of two enantiomers are also included within the invention.

Also included are solvates and isotopically-labelled peptides. Isotopically-labelled peptides are identical to the peptides recited herein, but for the fact that one or more atoms are replaced by an atom having an atomic mass or mass number different from the atomic mass or mass number predominantly found in nature. Examples of isotopes that can be incorporated into peptides of the invention include isotopes of hydrogen, carbon, nitrogen, oxygen and sulfur, such as ²H, ³H, ¹³C, ¹⁴C ¹⁵N, ¹⁸O, ¹⁷O, and ³⁵S, respectively.

Prodrugs of the peptide are also within the scope of the invention. Upon administration to a subject, a prodrug undergoes conversion by metabolic or other chemical processes to yield the peptide of the invention.

All amorphous and crystalline forms of the peptide of the invention are included.

The peptide of the invention may be administered alone. In some embodiments, the peptide of the invention is administered as part of a pharmaceutical composition. Such a pharmaceutical composition comprises an effective amount of the peptide of the invention, in combination with one or more pharmaceutically acceptable excipients. The excipient may aid transport of the peptide of the invention to the site in the body where it is intended to act, for example by increasing the rate of dissolution of the compound into the blood stream or by increasing the stability of the compound in order to delay its release, in order to increase its efficiency and prevent damage to tender tissues. Alternatively, the excipient may be for identification purposes, or to make the compound more appealing to the patient, for example by improving its taste, smell and/or appearance. Typically, the excipient makes up the bulk of the pharmaceutical composition.

Excipients include diluents or fillers, binders, disintegrants, lubricants, colouring agents and preservatives. Diluents or fillers are inert ingredients that may affect the chemical and physical properties of the final composition. If the dosage of the peptide is small then more diluents will be required to produce a composition suitable for practical use. If the dosage of the peptide is high then fewer diluents will be required.

Binders add cohesiveness to powders in order to form granules, which may form a tablet. The binder must also allow the tablet to disintegrate upon ingestion so that the peptide dissolves. Disintegration of the composition after administration may be facilitated through the use of a disintegrant.

An extensive overview of pharmaceutically acceptable excipients is described in the Handbook of Pharmaceutical Excipients, 6th Edition; Editors R. C. Rowe, P. J. Sheskey and M. E. Quinn, The Pharmaceutical Press, London, American Pharmacists Association, Washington, 2009. Any suitable pharmaceutically acceptable excipient is within the scope of the invention.

Pharmaceutical compositions include those suitable for intravenous, intramuscular, intrathecal, intraosseous, subcutaneous and oral administration, injection directly into a fracture, administration directly to the bone cells or the surrounding media and administration by implant. In some embodiments, the pharmaceutical composition is suitable for intravenous, intramuscular, intrathecal, and subcutaneous administration, injection directly into a fracture, administration directly to the bone cells or the surrounding media and administration by implant.

The pharmaceutical compositions of the invention may be compressed into solid dosage units, such as tablets, or be processed into capsules or suppositories. Preferably, the pharmaceutical compositions are injected and are prepared in the form of a solution, suspension or emulsion for such. Alternatively, the pharmaceutical compositions may be administered as a spray. Otherwise, the pharmaceutical compositions of the invention may be processed into an implant or any other preparation for immediate and/or sustained release.

Typically, the pharmaceutical compositions are processed into a solution, suspension or emulsion for intravenous, intramuscular and intrathecal administration, injection directly into a fracture, administration directly to the bone cells or the surrounding media and administration by implant.

When the peptide of the invention is used for the manufacture of a medicament, such a medicament includes any substance used for medical treatment. For the avoidance of doubt, implants lie within the definition of a medicament. Also contemplated within the scope of a medicament is a scaffold structure to which bone cells or their precursors are attached. Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention as described herein without departing from the scope of the invention as described. The present embodiments are therefore to be considered for descriptive purposes, are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The aspects and embodiments of the invention are further described in the following clauses:
1. A method of reducing bone loss and/or stimulating bone production, the method comprising administering an effective amount of a peptide comprising the amino acid sequence SVTEQGAELSNEER (SEQ ID NO:1), or variants thereof, to a patient and/or bone cells and/or their precursors.
2. The method of clause 1 wherein the method is of stimulating bone production.
3. The method of clause 1 or clause 2 wherein the peptide is administered ex vivo directly to the bone cells or their precursors or surrounding media.
4. The method of any one of clauses 1 to 3 wherein the bone cells are osteoblasts or osteoblast precursors.
5. The method of any one of clauses 1 to 4 wherein the bone cells are osteoblasts.
6. The method of clause 5 wherein the osteoblasts are primary osteoblasts.
7. The method of clause 5 or clause 6 wherein the osteoblasts are mammal osteoblasts.
8. The method of clause 5 or clause 6 wherein the osteoblasts are human osteoblasts.
9. The method of any one of clauses 3 to 8 further comprising transplanting the bone cells into a patient.
10. The method of clause 9 wherein the patient requires treatment and/or prophylaxis of musculoskeletal loss and/or damage.
11. A method of treatment and/or prophylaxis of musculoskeletal loss and/or damage in a patient, the method comprising administering an effective amount of the peptide of clause 1.
12. The method of clause 10 or clause 11 wherein the musculoskeletal loss and/or damage is associated with osteoporosis and/or bone injury.
13. The method of clause 12, wherein the osteoporosis results from any one or a combination of the group consisting of aging; prolonged bed rest; space travel; genetic disorders including cystic fibrosis, Ehlers-Danlos, glycogen storage diseases, Gaucher's disease, homocystinuria, hypophosphatasia, idiopathic hypercalciuria, Marfan syndrome, Menkes steely hair syndrome, osteogenesis imperfect, porphyria and Riley-Day syndrome; hypogonadal states including androgen insensitivity, anorexia nervosa, athletic amenorrhea, hyperprolactinemia, panhypopituitarism, premature ovarian failure and Turner's and Klinefelter's syndrome; endocrine disorders including acromegaly, adrenal insufficiency, Cushing's Syndrome, Diabetes Mellitus (Type 1), hyperparathyroidism and thyrotoxicosis; gastrointestinal diseases including gastrectomy, inflammatory bowel disease, malabsorption, celiac disease and primary biliary cirrhosis; hematologic disorders including hemophilia, leukemias and lymphomas, multiple myeloma, sickle cell disease, systemic mastocytosis and thalassemia; rheumatic and auto-immune diseases including ankylosing spondylitis, lupus and rheumatoid arthritis; alcoholism; amyloidosis; chronic metabolic acidosis; congestive heart failure; depression; emphysema; end stage renal disease; epilepsy; idiopathic scoliosis; immobilization; multiple sclerosis; muscular dystrophy; post-transplant bone disease; and sarcoidosis.
14. The method of clause 12 wherein the osteoporosis results from any one or a combination of the group consisting of aging, prolonged bed rest, anorexia nervosa, Diabetes Mellitus (Type 1), hyperparathyroidism, inflammatory bowel disease, malabsorption, celiac disease, hemophilia, leukemias and lymphomas, multiple myeloma, lupus, rheumatoid arthritis, alcoholism, depression, emphysema, epilepsy, immobilization, multiple sclerosis, muscular dystrophy and post-transplant bone disease.
15. The method of clause 12 wherein the osteoporosis results from any one or a combination of the group consisting of aging, prolonged bed rest, anorexia nervosa, hyperparathyroidism, hemophilia, leukemia's and lymphomas, multiple myeloma, alcoholism, depression, emphysema, epilepsy, immobilization, muscular dystrophy and post-transplant bone disease
16. The method of clause 12, wherein the osteoporosis results from aging.
17. The method of any one of clauses 12 to 16, wherein the bone injury is associated with sports injuries or any one or a combination of neurological disorders including stroke, multiple sclerosis, cerebral palsy, Parkinson's disease, spinal cord injury, neuropathy, sciatica and dementia; delirium; dizziness; vertigo; and dehydration.
18. The method of clause 12, wherein the musculoskeletal loss and/or damage is associated with age-related osteoporosis.
19. The method of any one of clauses 10 to 18 wherein the musculoskeletal loss and/or damage is bone fracture.
20. The method of any one of clauses 1, 2 and 9 to 19 wherein the patient is a mammal.
21. The method of any one of clauses 1, 2 and 9 to 19 wherein the patient is any one selected from the group consisting of human, horse, dog, cattle, sheep, pig and cat.
22. The method of any one of clauses 1, 2 and 9 to 19 wherein the patient is a human.
23. The method of any one of clauses 1, 2 and 11 to 22 wherein the peptide is administered by any one of the methods consisting of intravenous, intramuscular, intrathecal, intraosseous, subcutaneous and oral administration, injection directly into a fracture, administration directly to the bone cells or surrounding media and administration by implant.
24. The method of any one of clauses 1, 2 and 11 to 22 wherein the peptide is administered by any one of the methods consisting of intravenous, intramuscular, intrathecal and subcutaneous administration, injection directly into a fracture, administration directly to the bone cells or surrounding media and administration by implant.
25. The method of clause 23 or clause 24 wherein the implant allows for slow release of the peptide.
26. The method of any one preceding clause wherein the peptide consists of the amino acid sequence SVTEQGAELSNEER (SEQ ID NO:1), or variants thereof.
27. The method of any one of clauses 1 to 25 wherein the peptide consists essentially of the amino acid sequence SVTEQGAELSNEER (SEQ ID NO:1).
28. The method of any one of clauses 1 to 25 wherein the peptide consists of the amino acid sequence SVTEQGAELSNEER (SEQ ID NO:1).
29. A composition comprising the bone cells of any one of clauses 4 to 8 and the peptide of any one of clauses 1 and 26 to 28.
30. An orthopedic implant comprising an effective amount of the peptide of any one of clauses 1 and 26 to 28.
31. The orthopedic implant of clause 30, wherein peptide release is sustained.
32. The orthopedic implant of clause 30 or 31 wherein the peptide is within a coating.
33. The orthopedic implant of clause 30 or clause 31 comprising orthopaedic hardware coated with the peptide.
34. A composition comprising bone cement and an effective amount of the peptide of any one of clauses 1 and 26 to 28.
35. The composition of clause 34 wherein the bone cement is selected from any one of the group consisting of polymethyl methacrylate, calcium phosphate cement and glass polyalkenoate cement.
36. Use of the peptide of any one of clauses 1 and 26 to 28 for the method of any one of clauses 1 to 25.
37. Use of the peptide of any one of clauses 1 and 26 to 28 for the manufacture of a medicament for the method of any one of clauses 1 to 25.
38. A peptide of any one of clauses 1 and 26 to 28 for use in a method according to any one of clauses 1 to 25.

The following are presented as non-limiting examples of the invention.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

In the examples below, it is shown that the peptides of the invention, and the associated methods and uses, are surprisingly effective in reducing bone loss and/or stimulating bone production when administered to a patient and/or bone cells in effective amounts.

### Example 1: General Information

Alta Bioscience (University of Birmingham, Birmingham, UK) synthesized PEPITEM.

Osteoclastogenic/differentiation media for osteoclasts is made up of Minimum Essential Medium (MEM) Eagle (Sigma, M8042), supplemented with 10% fetal calf serum (FCS) and 1% guinea pig serum (GPS), with addition of 50 ng/ml receptor activator of nuclear factor-κB ligand (RANKL) (R&D—462-TEC) and 50 ng/ml macrophage colony-stimulating factor (m-CSF) (R&D, 416-ML).

Osteoblast differentiation media is made up of MEM, FCS and GPS supplemented with 10 mM B-Glycerophosphate (Sigma—G9422) and 50 ug/ml L-Ascorbic acid (Sigma, A5960).

### Example 2. Methodology

### A. In Vitro Assays

### B. Cells

1. MC3T3-E1 murine osteoblast cell line;
2. Murine stromal ST2 cell line;
3. Primary murine osteoblasts isolated from the calvarias of pups;
4. Primary human osteoblasts obtained from patients undergoing joint replacement surgery for osteoarthritis;
5. Osteoclasts cultured from osteoclast precursors isolated from tibia and femur bone marrow of mice

### C. MC3T3-E1 Cells

The spontaneously immortalised murine MC3T3-E1 cell line (ATCC, England, UK, CRL-2593) was brought up from liquid nitrogen and cultured in basal media made up of: αMEM media (Sigma-Aldrich™, St. Louis, Mo., USA, M8042) supplemented with 2 mM L-Glutamine, 100 U/ml Penicillin and 100 μg/ml Streptomycin (all from Sigma-Aldrich™, G1146) and fetal bovine serum [(FBS) Biosera™, East Sussex, UK, FB1001]. MC3T3-E1 cells (8×10³ cells/well) were seeded into a 48-well plate and were treated with or without 10 ng/ml PEPITEM (Alta Bioscience™; Redditch, UK) diluted in differentiation media consisting of Basal media supplemented with 10 mM β-Glycerol phosphate (Sigma-Aldrich, G9422) and 50 μg/ml L-Ascorbic acid (Sigma-Aldrich™, A5960). Cells were cultured for up to 21 days at 37° C. in 5% CO₂, with media being changed every other day. As a control, cells were cultured in basal media. Mineralisation was assessed using an Alizarin red staining quantification assay and ALP activity.

### D. ST2 Cells

The murine stromal ST2 cell line was provided by Dr James Edwards, University of Oxford and brought up from liquid nitrogen in ST2 media containing: RPMI-1640 media (Sigma-Aldrich™, R8758) supplemented with 2 mM L-Glutamine, 100 U/ml Penicillin and 100 μg/ml Streptomycin (all from Sigma-Aldrich™, G1146) and fetal bovine serum [(FBS) Biosera™, FB1001]. ST2 cells (8×10³ cells/well) were seeded into a 12 well plate and were treated with or without 10 ng/ml PEPITEM (Alta Bioscience™) diluted in differentiation media consisting of ST2 media supplemented with 2 mM β-Glycerol phosphate (Sigma-Aldrich™, G9422) and 50 m/ml L-Ascorbic acid (Sigma-Aldrich^{TM}, A5960). Cells were cultured for 4 days at 37° C. in 5% CO₂, with media being changed every other day. As a control, cells were cultured in ST2 media. Mineralization was assessed using an ALP activity assay.

### E. Primary Calvarial Osteoblasts

Day 3-5 WT mouse pups were culled via decapitation and heads were washed briefly in 70% ethanol and kept hydrated in αMEM media supplemented with 2 mM L-Glutamine, 100 U/ml Penicillin and 100 μg/ml Streptomycin. Skin was removed from around calvaria using forceps, and the calvaria was dissected out and cleaned of any contaminating tissue. Calvaria were added to αMEM media supplemented with 1 mg/ml of collagenase d (Roche™, Basel, Switzerland, COLLD-RO). Tubes were then agitated at 37° C. for 10 minutes and the media discarded. αMEM media supplemented with 1 mg/ml of collagenase d was again added to calvaria and shaken at 37° C. for 30 minutes. Media (containing cells) was then removed and kept on ice Media (containing cells) was then removed and kept on ice. The calvaria were then again shaken at 37° C. for 10 minutes in 1.5 ml of αMEM media supplemented with 5 μM EDTA (Sigma-Aldrich™, E7889) and the cell containing media transferred to the sample on ice, pooling the cells. Finally, the calvaria was incubated with 1 mg/ml collagenase d in αMEM and agitated at 37° C. for 30 minutes, before cell containing media was pooled with the cells on ice collected from the 2 previous wash steps. Cells on ice were then spun down at 300 g for 4 minutes, resuspended in 1 ml of Basal media and counted using a haemocytometer. Cells were then diluted to 1×10⁶ cells/ml and split among culture flasks with additional Basal media. After 24 hours, media was changed to remove non-adhesive and contaminated cells. Primary calvarial osteoblasts were then utilised at P1, or frozen down for analysis later.

### F. Human Osteoblasts

Human osteoarthritis subchondral joint tissue was obtained at the time of total knee and hip joint replacement operations from the Royal Orthopaedic Hospital (Birmingham). To isolate primary osteoblasts, cartilage was removed from the femoral condyles and tibial plateaus and cut into 2 mm² pieces. Samples were kept in media and cleaned of fat. Media contains DMEM (Sigma™, D6546), FCS (10%), GPS (1%), Non-essential amino acid (1%; Sigma™, M7145), β-glycerophosphate (2 mM; Sigma™—G9422) and L-Ascorbic acid (50 μg/ml; Sigma™, A5960). Bone chips were placed in T75 flasks in 10 ml of media, replacing media every 2-3 days. Main outgrowth occurs between 10-14 days, after which chips were placed in new flasks. At 90% confluence, cells were trypsinised and used or split for further culture.

### G. Effect of Peptide on Osteoblast Cells

Osteoblasts were cultured in mineralization differentiation media in the presence or absence of PEPITEM (10 ng/ml) over a 21-day period, dependent on cell source. Mineralization was assessed by quantifying the amount of alizarin red (nM) or the level of alkaline phosphate activity (absorbance at 405 nm) per condition.

### H. Alizarin Red Mineralization Assay

Alizarin red analysis was performed following the manufacturer's instructions (Caltag+Medsystems™, Buckingham, UK, SC8678). Briefly, cells were washed twice in PBS and fixed in 4% paraformaldehyde for 15 minutes at room temperature. Cells were washed a further 3 times in distilled H₂O and stained with 40 mM of Alizarin red S (ARS) for 35 minutes at room temperature with gentle shaking on a digital orbital shaker (Heathrow Scientific™) Following staining, excess dye was removed by washing the wells at least 5 times with deionised H₂O until the water was clear. Cells were imaged using the Cytation™ 5 microscope (Biotek™; VT, USA), with digitization of 20 random fields of view in the middle of the well. Images per well were merged to produce a region of interest per well. Alizarin red was quantified using ImageJ™ (NIH) by removing backing, converting to a binary image and measuring area fraction stained. Data were presented as the percentage of image stained.

Alizarin red stain was quantified by dye extraction as per manufacturer's instructions (ScienceCell™, San Diego, Calif., 8678); cells were treated with 10% acetic acid for 30 minutes at room temperature with gentle shaking on a digital orbital shaker and then collected into a 1.5 ml centrifuge tube (CoStar™) using a cell scraper (Sarstedt™, Nümbrecht, Germany, 83.1830). Cells were then vortexed, heated to 85° C. for 10 minutes in a Sub Aqua Pro water bath (Grant™, Cambridge, UK, SAP26) and cooled on ice for 5 minutes. Tubes were then centrifuged at 20,000 g for 15 minutes, the supernatant collected and then neutralized to pH 4.1-4.5 with 10% ammonium hydroxide. Neutralized supernatants were transferred to a 96-well plate, and absorbance at 405 nm was read by a Synergy™ HT plate reader (BioTek™). Each sample was run in technical replicates which were used to calculate a mean. A standard curve was produced by measuring the absorbance of Alizarin red standard from a concentration of 0.0313 mM to 2 mM and using the equation of the standard curve, the concentration of Alizarin red was calculated. Data were expressed as concentration of Alizarin red in each well as a percentage of differentiation media control.

### I. Alkaline Phosphatase Assay

To measure alkaline phosphatase activity, cells were washed in PBS before being lysed in 100 μl of RIPA buffer (Thermo Fisher™, R0278) for 15 minutes on ice. Cells were harvested using a cell scraper, vortexed and centrifuged at 13000 g for 10 minutes. From the cell lysates, 10 μL was added to 90 μL of alkaline phosphatase yellow (pNPP) liquid substrate for ELISA (Sigma-Aldrich™, P7998) in a 96 well plate. The plate was wrapped in foil and left to run for 45 minutes at 37° C. in an incubated shaker (SciQuip™; Shropshire, UK) before being quantified using a Synergy HT plate reader (BioTek™) with absorbance set at 405 nm.

### J. In Vitro Osteoclast Culture

Eight-week-old C57B1/6 mice were culled and hind limbs were removed and cleaned of muscle and fat. Small cuts (˜1 mm) were made at both ends of the tibias and femurs before they were placed in 200 μl pipette tips inside a 1.5 ml Eppendorf™. Murine basal media was added to each Eppendorf™ before centrifugation at 10,000 g for 15 seconds to spin out the bone marrow from the inside of the bones. A 25 G needle was used to break up the resulting pellet, which was then suspended in 10 ml of murine basal media, filtered through a 70 μm pore (Greiner Bio-one™; cat: 542070, Kremsmünster, Austria) and counted using a hemocytometer.

In a 24-well osteoassay plate (Corning™, Amsterdam, The Netherlands), 1×10⁶ cells were cultured in 500 μl of murine basal media for 72 hours, to allow mononuclear cells to adhere before media was changed. Once 80-90% confluence had been reached, the media was changed to murine osteoclastogenic media with or without 10 ng/ml of PEPITEM. Media was replaced every 3 days, with differentiation occurring around day 7 seen by presence of large multi-nucleated cells.

### K. Analysis of Pits Within Wells of Osteoassay Plate

Resorption by osteoclasts was analyzed by assessing pit formation on the surfaces of wells of osteoassay plates coated with hydroxyapatite. 10% hydrogen peroxidase (H₂O₂, Sigma-Aldrich™; cat: H1009) was added for 5 minutes to remove all attached cells. Peroxidase was washed off with PBS, which was also removed and wells allowed to air dry. Once dry, wells were imaged on a Cytation™ 5 microscope to generate images of whole wells. Images were then opened in ImageJ™ and the color threshold was manually changed to distinguish and select pits. Pit areas were measured using the measurement tool and resorption calculated as a percentage of total area of the well.

### L. In Vivo Models and Analysis

All experiments were performed in accordance with UK Home Office regulations. For the animal studies, in each experiment wild-type (WT) animals were allocated at random to different experimental groups. Importantly, mice from the same litter were randomly distributed amongst the experimental groups, and where possible, they were equally distributed amongst experimental groups. Injections of peptide were carried out at the same time of day, with identical reagents when possible. The investigators were not blinded to allocation during experiments and outcome assessment. All samples analyzed were included in the study.

WT mice were housed in the Biomedical Services Unit at the University of Birmingham. Mice were used between 6 and 16 weeks of age.
1. Homeostasis—Six-week-old male wild-type mice were treated with or without daily intraperitoneal (i.p.) injections of PEPITEM (300 μg) for 2 weeks. Mice were culled at 2 weeks to examine the effect of PEPITEM on bone homeostasis in young resting bones.
2. Ovariectomy (model of age-related osteoporosis)—Twelve-week-old female wild-type mice were subjected to ovariectomy (OVX). Two weeks post-surgery, mice were treated with or without daily i.p. injections of PEPITEM (300 μg) for 2 weeks. Mice were culled at 2 weeks to obtain baseline bone loss measurements, or at 4 weeks to examine the effect of PEPITEM on OVX-induced bone loss.
3. Analysis of murine bone parameters—Histomorphometry analysis was performed on long bones (femur; tibia) and/or spine to determine the effects of PEPITEM on the structural architecture of the tissue, cellular distribution and bone volume, including TRAP and Toluidine blue staining for osteoclasts and osteoblasts respectively. For information on these staining methods, see Das, P. et al., PNAS, 2018, 115(43), E10137-E10146 and Waddington, R. J. and Sloan, A. J. eds. Tissue Engineering and Regeneration in Dentistry: Current Strategies. West Sussex: John Wiley & Sons. Femurs were subjected to a 3-point bending test using a Bose 5500 (Bose) to determine the stiffness of the bones and the force required to fracture the bone. For more information on the 3-point bending test method, see Huesa, C. et al., Bone, 2011, 48(5), 1066-1074.

### M. Tartrate-Resistant Acidic Phosphatase (TRAP) Staining

One tibia per mouse was decalcified for 4 weeks with 10% w/v ethylenediaminetetraacetic acid (EDTA; Sigma-Aldrich™, Cat: T4174) diluted in ultra-pure water and buffered to a pH of 7.4 using NaOH or HCL. Decalcified bones were sent to the Royal Orthopedic Hospital (ROH) Birmingham to be paraffin embedded and sectioned (10 sections per bone starting at the midpoint). In order to analyze the number of osteoclasts in each section, tartrate-resistant acidic phosphatase (TRAP) staining was performed. TRAP basic incubation medium was first made up by adding sodium acetate anhydrous (0.11 mM, VWR, cat: 10236), L+ Tartaric acid (0.074 mM, Fisher Scientific, Loughborough, UK, cat: 137855000) and glacial acetic acid (2%, Sigma-Aldrich™; cat: 1005706) to deionized water (diH₂O). TRAP staining solution was then made: 98% (V/V) TRAP basic incubation mix; 1.6 M Fast Red Violet LB salt (Sigma-Aldrich™, Cat:F3381); and 2% (V/V) of Naphthol AS-MX phosphate substrate mix (58 mM Naphthol AS-MX (Sigma-Aldrich™, Cat: N4875) in 2-ethoxyethanol (Alfa Aesar™, Lancashire, UK; cat: 16100)). TRAP staining solution was heated to 37° C. on the digital orbital shaker (Heathrow Scientific™; Illinois, USA). Slides were deparaffinized using Xylene (VWR; cat: 28975.325), followed by rehydration in decreasing concentrations of ethanol (100%, 90%, 80%, 70%; VWR, cat: 20821) and finished with two washes with diH₂O. Deparaffinized slides were then submerged in the TRAP staining solution for 30 minutes at 37° C. Stained slides were then mounted using immu-mount (ThermoFisher™ Scientific, Cat: 1900331) and covered with a coverslip. Slides were then imaged using the slide scanner axioscan Z1 (ZEISS; Oberkochen, Germany) and stored as .Zen files.

For analysis of whole hind limb sections, Zen Blue software (ZEISS, Cambridge, UK) was used to break whole leg scans into regions of 20 μm by 20 μm, which were saved as images (JPEG) and opened on ImageJ^{TM} (National institute of health (NIH); Maryland, USA). Images were then color separated in ImageJ using CIELAB colour space (L*A*B*), where L* (lightness) and B* (blue/yellow) were set as 0 to remove colors that are not red. A threshold was then manually set on A* (green/magenta) so that the image only showed pink osteoclasts. Osteoclasts were counted per region using ImageJ™ count maxima tool, where maxima were defined according to noise tolerance to neighboring squares and given as total number of osteoclasts per section. Manual counting and automated counting showed no differences in number of osteoclasts observed.

For analysis of tibias, samples were manually counted by drawing a ROI around the diaphysis, which was measured and exported to ImageJ™. Osteoclasts were counted manually using the ImageJ™ cell counter plugin, normalized to the area of the ROI and presented as osteoclasts/mm². Line measurements were taken for the line of chondroclasts, on which chondroclasts were counted and normalized to line length. Chondroclast measurements were presented as chondroclasts/m.

### Example 3. Discussion of Results

The data presented in FIG. 1 and FIG. 2 show that PEPITEM stimulates the production of bone mineral by murine cells (both murine osteoblast cell line MC3T3 and primary murine cells) and primary human osteoblasts when the cells are cultured in isolation in vitro. Murine osteoblast cell line MC3T3 (see FIG. 1E), primary murine osteoblast cells (see FIG. 1F) or primary human osteoblasts (see FIG. 1G) were allowed to mineralize over 21 days in the presence or absence of PEPITEM. Mineralization was assessed by quantification of Alizarin Red staining in murine osteoblasts using colorimetric spectrometry (images shown in FIG. 1A and in FIG. 1B and FIG. 1C) or by quantification of Alkaline Phosphatase Activity in human osteoblasts (FIG. 1D). PEPITEM significantly increased murine and human primary osteoblast mineralization.

Moreover, the data presented in FIG. 2 and FIG. 3 show that PEPITEM significantly increases bone formation over two weeks in long bones and vertebra of young healthy resting mice, when compared to control treated mice. Young, healthy WT mice were given daily injections with either PBS or PEPITEM for 2 weeks. MicroCT images were obtained from the long bones or vertebra. PEPITEM significantly increased the bone volume to trabecular bone volume ratio (BV/TV); trabecular number and trabecular thickness with respect to bone treated with PBS. PEPITEM also decreased the trabecular separation (in line with an increase in trabecular number), indicating that the trabecular are closer together and that there is more bone. Crucially the differences observed with PEPITEM are comparable to bisphosphonate treatments given over >4-week timeframe. Thus, PEPITEM increases bone formation in young bones.

The data presented in FIG. 4 show that this increase in trabecular bone volume is mirrored by an increase in the strength of the bone and the forces required to cause the bone to bend and eventually break. Young, healthy WT mice were given daily injections with either PBS or PEPITEM for 2 weeks. Long bones were subject to a 3-point bending test to measure the stiffness of the bone, the force required to induce the bone to bend and the force required to completely fracture/break the bone. PEPITEM significantly increases the strength of the bone over a two-week treatment period compared to PBS treated mice. Thus, PEPITEM increases bone strength in young bones.

In addition, the data presented in FIG. 5 show that PEPITEM treatment halts further bone loss induced by ovariectomy. Young, healthy WT mice were subjected to ovariectomy. After 2 weeks mice were culled for baseline bone analysis, left untreated for 2 weeks or given daily injections with PEPITEM for 2 weeks. MicroCT images were obtained from the long bones either 2- or 4-weeks post ovariectomy. PEPITEM significantly increased the bone volume to trabecular bone volume ratio (BV/TV); trabecular number and trabecular thickness, and decreased trabecular separation. Thus, PEPITEM prevents age-related bone loss in a model of age-related osteoporosis.

The results strongly indicate that PEPITEM acts directly on the cells that make bone (osteoblasts) causing them to increase trabecular bone formation, strengthening the bone and making it more resistant to erosion and fracture. Moreover, the results support that PEPITEM can limit bone loss caused by age-related osteoporosis.

The data presented in FIG. 6 show that daily injections of PEPITEM to young, healthy wild-type mice for 14 days significantly reduces the number of osteoclasts per mm² within the tibia of such mice. The number of chondroclasts per μm also reduced with respect to the mice injected with PBS. Interestingly, the data shown in FIG. 7 indicate that the resorption activity of murine osteoclasts cultured in wells comprising 10 ng/ml of PEPITEM is lower than osteoclasts cultured without PEPITEM. Thus, the results indicate that PEPITEM has anti-osteoclastogenesis properties, reducing osteoclast number and activity in vitro and in vivo.

Collectively these results strongly indicate that PEPITEM has dual actions—it is able to stimulate the activity of osteoblasts to trigger bone production whilst simultaneously being able to inhibit the activity of osteoclasts to limit bone loss.

1. A method of reducing bone loss and/or stimulating bone production, the method comprising administering an effective amount of a peptide comprising the amino acid sequence SVTEQGAELSNEER (SEQ ID NO:1), or variants thereof, to a patient in need thereof, bone cells, bone cell precursors, or a combination thereof.

2. The method of claim 1 wherein the method stimulates bone production.

3. The method of claim 1 wherein the peptide is administered ex vivo directly to the bone cells, bone cell precursors, surrounding media, or a combination thereof.

4. The method of claim 1 wherein the bone cells are osteoblasts.

5. The method of claim 4 wherein the osteoblasts are primary osteoblasts.

6. The method of claim 4 wherein the osteoblasts are mammal osteoblasts.

7. The method of claim 4 wherein the osteoblasts are human osteoblasts.

8. The method of claim 3 further comprising transplanting the bone cells into a patient.

9. The method of claim 8 wherein the patient requires treatment and/or prophylaxis of musculoskeletal loss and/or damage.

10. A method of treatment, prophylaxis, or both of musculoskeletal loss or damage in a patient in need thereof, the method comprising administering an effective amount of SVTEQGAELSNEER (SEQ ID NO:1), or variants thereof.

11. The method of claim 10 wherein the musculoskeletal loss or damage is associated with osteoporosis, bone injury, or both.

12. The method of claim 11 wherein the osteoporosis results from any one or a combination of the group consisting of aging, prolonged bed rest, anorexia nervosa, Diabetes Mellitus (Type 1), hyperparathyroidism, inflammatory bowel disease, malabsorption, celiac disease, haemophilia, leukemias and lymphomas, multiple myeloma, lupus, rheumatoid arthritis, alcoholism, depression, emphysema, epilepsy, immobilisation, multiple sclerosis, muscular dystrophy and post-transplant bone disease.

13. The method of claim 11 wherein the bone injury is associated with sports injuries or any one or a combination of neurological disorders including stroke, multiple sclerosis, cerebral palsy, Parkinson's disease, spinal cord injury, neuropathy, sciatica and dementia; delirium; dizziness; vertigo; and dehydration.

14. The method of claim 10 wherein the musculoskeletal loss and/or damage is bone fracture.

15. The method of claim 1 wherein the patient is a mammal.

16. The method of claim 10 wherein the patient is a mammal.

17. The method of claim 15 wherein the mammal is a human.

18. The method of claim 16 wherein the mammal is a human.

19. The method of claim 1 wherein the peptide is administered by a method selected from the group consisting of intraveneous, intramuscular, intrathecal and subcutaneous administration, injection directly into a fracture, administration directly to the bone cells or surrounding media and administration by implant.

20. The method of claim 10 wherein the peptide is administered by a method selected from the group consisting of intraveneous, intramuscular, intrathecal and subcutaneous administration, injection directly into a fracture, administration directly to the bone cells or surrounding media and administration by implant.

21. A composition comprising an effective amount of the peptide SVTEQGAELSNEER (SEQ ID NO:1) or a variant thereof and bone cells or bone cell precursors.

22. An orthopedic implant comprising an effective amount of the composition of claim 21.

23. A composition comprising bone cement and an effective amount of the composition of claim 21.


SEQUENCE LISTING (1 sequence)

SEQ ID NO: 1 - protein, 14 aa, Artificial Sequence
  PEPITEM
SVTEQGAELSNEER